(19) United States
(12) Patent Application Publication
(10) Pub. No.: US 2011/0166522 A1
(43) Pub. Date: Jul. 7, 2011

(54) ACCUMULATOR FOR THERAPEUTIC FLUID DELIVERY DEVICES

(75) Inventors: James M. Haase, Maplewood, MN (US); Jeffrey P. Bodner, St. Paul, MN (US)

(73) Assignee: Medtronic, Inc., Minneapolis, MN (US)

(21) Appl. No.: 12/826,115

(22) Filed: Jun. 29, 2010

Related U.S. Application Data
(60) Provisional application No. 61/292,655, filed on Jan. 6, 2010.

(51) Int. Cl. A61M 31/00; A61M 5/142

(52) U.S. Cl. 604/151; 604/288.01

(57) ABSTRACT

A therapeutic fluid delivery device includes a flexible member configured as an accumulator for flow of a therapeutic fluid through the fluid delivery device to a patient. In one example, the flexible member includes a catheter access port (CAP) septum.

This application claims the benefit of U.S. Provisional Application No. 61/292,655, filed Jan. 6, 2010, the entire content of which is incorporated herein by this reference.

### TECHNICAL FIELD

This disclosure relates generally to implantable medical devices and, more particularly, to implantable fluid delivery devices.

### BACKGROUND

A variety of medical devices are used for chronic, i.e., long-term, delivery of fluid therapy to patients suffering from a variety of conditions, such as chronic pain, diabetes, tremor, Parkinson's disease, epilepsy, urinary or fecal incontinence, sexual dysfunction, obesity, spasticity, or gastroparesis. For example, pumps or other fluid delivery devices can be used for chronic delivery of therapeutic agents, such as drugs to patients. These devices are intended to provide a patient with a therapeutic output to alleviate or assist with a variety of conditions. Typically, such devices are implanted in a patient and provide a therapeutic output under specified conditions on a recurring basis.

One type of implantable fluid delivery device is a drug infusion device that can deliver a fluid medication to a patient at a selected site. A fluid delivery device, such as a drug infusion device, may be implanted at a location in the body of a patient and deliver a therapeutic fluid through a catheter to a selected delivery site in the body. Fluid delivery devices, such as implantable drug pumps, commonly include a reservoir for holding a supply of the therapeutic fluid, such as a drug or other substance, for delivery to a site in the patient. The fluid reservoir can be self-sealing and accessible through one or more ports. A pump is fluidly coupled to the reservoir for delivering the therapeutic fluid to the patient. A catheter provides a pathway for delivering the therapeutic fluid from the pump to the delivery site in the patient.

### SUMMARY

In general, this disclosure is directed to a flexible member arranged in the flow path of a therapeutic fluid delivery device. The flexible member is configured as an accumulator for flow of a therapeutic fluid through the device to a patient. In one example, the flexible member comprises a catheter access port (CAP) septum.

In one example, an implantable fluid delivery system includes a fluid delivery device and a catheter access port (CAP) septum. The fluid delivery device is configured to deliver a therapeutic fluid to a patient. The catheter access port (CAP) septum is connected to the fluid delivery device and configured as an accumulator for flow of the therapeutic fluid through the fluid delivery device.

In another example, a catheter access port (CAP) septum for a therapeutic fluid delivery device includes a rim, a hub, and a web. The rim is configured to be secured to a structure and comprises a first thickness. The hub is surrounded by the rim and comprises a second thickness that is less than the first thickness of the rim. The web connects the rim to the hub and comprises a third thickness that is less than the first thickness of the rim and the second thickness of the hub.

In an additional example, a catheter access port (CAP) septum for a therapeutic fluid delivery device includes a disc and at least one material void. The disc comprises two circular major surfaces connected by a minor annular surface. The at least one material void is formed in at least one of the two circular major surfaces and is configured to facilitate deformation of the CAP septum under pressure from a therapeutic fluid in a flow path of the fluid delivery device.

In another example, a therapeutic fluid delivery device includes a pump, a processor, and a catheter access port (CAP) septum. The pump is configured to deliver a therapeutic fluid to a patient via a flow path. The processor is programmable to cause the pump to deliver the therapeutic fluid to the patient. The CAP septum is arranged along the flow path and includes at least one material void configured to facilitate deformation of the CAP septum under pressure from the therapeutic fluid in the flow path.

The details of one or more examples disclosed herein are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example of a fluid delivery system including an implantable fluid delivery device configured to deliver a therapeutic fluid to a patient via a catheter.

FIG. 2 is functional block diagram illustrating an example of the implantable fluid delivery device of FIG. 1.

FIG. 3 is a functional block diagram illustrating an example of an external programmer forming part of the fluid delivery system of FIG. 1.

FIG. 4 is a plan view of an example configuration of an implantable fluid delivery device including a catheter access port (CAP).

FIG. 5 is a section view of an example catheter access port of a fluid delivery device cut along section line A-A of FIG. 4.

FIGS. 6A and 6B are detail views of the catheter access port of FIGS. 4 and 5.

### DETAILED DESCRIPTION

Medical devices are useful for treating, managing or otherwise controlling various patient conditions or disorders including, e.g., pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, mood disorders, or other disorders. Some medical devices, referred to herein generally as fluid delivery devices, may be configured to deliver one or more fluid therapeutic agents, alone or in combination with other therapies, such as electrical stimulation, to one or more target sites within a patient. For example, in some cases, a fluid delivery device may deliver a pain-relieving drug to a patient with chronic pain, insulin to a patient with diabetes, or another fluid to a patient with a different disorder. The device may be implanted in the patient for chronic therapy delivery (i.e., longer than a temporary, trial basis) or temporary delivery.

The operation of fluid delivery devices may be defined by a number of parameters related to the amount and timing of therapeutic fluid delivery to a patient. In some examples, the therapeutic fluid delivery parameters are defined in a dosing or therapy program and/or therapy schedule. A dosing or therapy program generally may refer to a program sent to an implantable fluid delivery device by a programming device to cause the fluid delivery device to deliver fluid at a certain rate and at a certain time. The dosing program may include, for example, definitions of a priming bolus, a bridging bolus, a supplemental bolus, and a therapy schedule. A dosing program may include additional information, such as patient information, permissions for a user to add a supplemental bolus, as well as limits on the frequency or number of such boluses, historical therapy schedules, fluid or drug information, or other information.

A therapy schedule generally refers to a rate (which may be zero) at which to administer a fluid, or a drug or drug combination within the fluid, at specific times to a patient. In particular, the therapy schedule may define one or more programmed doses, which may be periodic or aperiodic including, e.g., a rate of fluid delivery and different times and/or time durations for which to deliver the dose. Dose generally refers to the amount of drug delivered over a period of time, and may change over the course of a therapy schedule such that a drug may be delivered at different rates at different times.

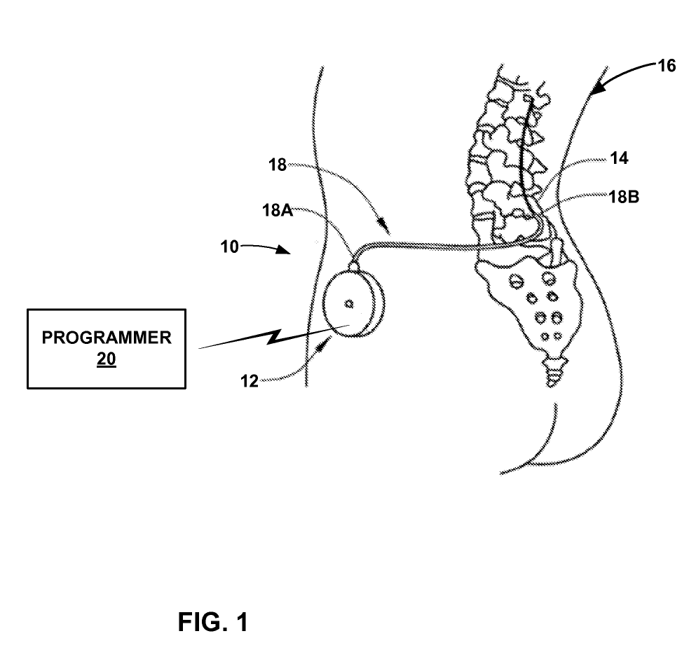

FIG. 1 is a conceptual diagram illustrating an example of a therapy system 10, which includes implantable medical device (IMD) 12, catheter 18, and external programmer 20. IMD 12 is connected to catheter 18 to deliver at least one therapeutic agent, such as a pharmaceutical agent, pain relieving agent, anti-inflammatory agent, biological agent, gene therapy agent, or the like, to a target site within patient 16. Example therapeutic agents that IMD 12 can be configured to deliver include, but are not limited to, insulin, morphine, hydromorphone, bupivacaine, clonidine, other analgesics, baclofen and other muscle relaxers and antispastic agents, genetic agents, antibiotics, nutritional fluids, hormones or hormonal drugs, gene therapy drugs, anticoagulants, cardiovascular medications or chemotherapeutics.

In the example of FIG. 1, IMD 12 delivers the therapeutic fluid to patient 16 through catheter 18. In particular, IMD 12 delivers the fluid from proximal end 18A coupled to IMD 12 to distal end 18B located proximate to the target site. Catheter 18 can comprise a unitary catheter or a plurality of catheter segments connected together to form an overall catheter length. External programmer 20 is configured to wirelessly communicate with IMD 12 as needed, such as to provide or retrieve therapy information or control aspects of therapy delivery (e.g., modify the therapy parameters such as rate or timing of delivery, turn IMD 12 on or off, and so forth) from IMD 12 to patient 16.

In accordance with the examples described in this disclosure, IMD 12 includes at least one flexible member that is configured as an accumulator for flow of a therapeutic fluid through the fluid delivery device to a patient. Generally, an accumulator is an energy storage device. In hydraulic systems, an accumulator functions to store or absorb pressure of a non-compressible fluid. As used in this disclosure, accumulator is any device in a fluid flow system that creates compliance in the flow path of the system such that the accumulator is capable of absorbing pressure exerted on it by a fluid flowing along the path. In the example of FIG. 1, the energy required to pump the therapeutic fluid from IMD 12 to patient 16 can be reduced by creating compliance in the flow path downstream of the pumping mechanism of the device. In this context, compliance means that some portion of the flow path is flexible or non-rigid. For example, the compliance may be sufficient to at least partially absorb the initial pressure spike induced by the delivery of a fluid pulse through the flow path by a pump within IMD 12. Reducing the energy requirements of IMD 12 increases device longevity by reducing the load on the pump and an internal power source that powers the pump, e.g., a battery of the device. The addition of a dedicated component to create compliance, however, may add cost and complexity to the device. The following examples, therefore, achieve compliance in the flow path of IMD 12 via design of a component that already exists in the device.

In one example, the flexible member of IMD 12 that is configured as an accumulator is a catheter access port (CAP) septum. Generally, the CAP septum is constructed from an elastomeric material. Examples of suitable elastomeric materials include, e.g., silicone, fluoroelastomers, and perfluoroelastomers. The CAP septum allows access to the flow path of the therapeutic fluid within IMD 12 via a hypodermic needle through the skin of patient 16. By forming the CAP septum in a particular shape, as is described in greater detail below, the membrane can be configured to deform under pressure and then recover from the deformation, e.g., like a trampoline. For example, the CAP septum may deform from a generally planar state to a bowed state in which surfaces of the septum deflect in a direction generally perpendicular to a major plane of the septum. Employing the CAP septum of IMD 12 as an accumulator has an additional advantage of reducing fatigue related failures, because the septum is formed from an elastomeric material that makes the septum generally less susceptible to the deleterious effects of cyclic loading than metallic accumulators commonly employed in fluid delivery devices. In addition, the shape and material composition of the CAP septum may be designed with sufficient radial compression to seal after being punctured by a needle, which ordinarily is a necessary attribute to allow percutaneous access to IMD 12 via the CAP.

Septum resealing is generally created by radial stresses inherent in the material from which the CAP septum is formed. The radial stresses are created by axial compression of the septum with a constrained periphery and are the result of Poisson's effect and the incompressible nature of the material from which the septum is formed. Deformation of the CAP septum, on the other hand, is facilitated by forming the septum with a material void that creates an offset between the septum and adjacent structure of IMD 12 such that the septum may deform in response to the pressure of the therapeutic fluid flowing through IMD 12.

In some examples, a CAP septum designed in accordance with the examples described in this disclosure may have a volumentric displacement in a range from approximately 0.1 to approximately 5 microliters (0.00000338 to 0.000169 ounces) for pressures in a range from approximately 3.5 to approximately 138 kilopascals. (0.5 to 20 pounds per square inch). Computer simulation models and prototype testing has confirmed that one CAP septum designed in accordance with the examples described in this disclosure may have a volumetric displacement of at least approximately 1 micro liters (0.0000338 ounces) under approximately 6.9 kilopascals (10 pounds per square inch) of pressure. In the context of this disclosure, volumetric displacement is the volume of therapeutic fluid that deformation of the CAP septum permits to be displaced at a given fluid pressure.

IMD 12, in general, may have an outer housing that is constructed of a biocompatible material that resists corrosion and degradation from bodily fluids. Examples of suitable biocompatible materials may include, e.g., titanium or biologically inert polymers. IMD 12 may be implanted within a subcutaneous pocket relatively close to the therapy delivery site. For example, in the example shown in FIG. 1, IMD 12 is implanted within an abdomen of patient 16. In other examples, IMD 12 may be implanted within other suitable sites within patient 16, which may depend, for example, on the target site within patient 16 for the delivery of the therapeutic agent. In still other examples, IMD 12 may be external to patient 16 with a percutaneous catheter connected between IMD 12 and the target delivery site within patient 16.

Catheter 18 may be coupled to IMD 12 either directly or with the aid of a catheter extension (not shown in FIG. 1). In the example shown in FIG. 1, catheter 18 traverses from the implant site of IMD 12 to one or more targets proximate to spine 14. Catheter 18 is positioned such that one or more fluid delivery outlets (not shown in FIG. 1) of catheter 18 are proximate to the targets within patient 16. In the example of FIG. 1, IMD 12 delivers a therapeutic agent through catheter 18 to targets proximate to spinal cord 14. IMD 12 can be configured for intrathecal drug delivery into the intrathecal space, as well as epidural delivery into the epidural space, both of which surround spinal cord 14. The epidural space (also known as “extradural space” or “peridural space”) is the space within the spinal canal (formed by the surrounding vertebrae) lying outside the dura mater, which encloses the arachnoid mater, subarachnoid space, the cerebrospinal fluid, and spinal cord 14. The intrathecal space is within the subarachnoid space, which is further inward past the epidural space and dura mater and through the theca.

Although the target site shown in FIG. 1 is proximate to spinal cord 14 of patient 16, other applications of therapy system 10 include alternative target delivery sites. The target delivery site in other applications of therapy system 10 can be located within patient 16 proximate to, e.g., sacral nerves (e.g., the S2, S3, or S4 sacral nerves) or any other suitable nerve, organ, muscle or muscle group in patient 16, which may be selected based on, for example, a patient condition. In one such application, therapy system 10 may be used to deliver a therapeutic agent to tissue proximate to a pudendal nerve, a perineal nerve or other areas of the nervous system, in which cases, catheter 18 would be implanted and substantially fixed proximate to the respective nerve. Positioning catheter 18 to deliver a therapeutic agent to various sites within patient 16 enables therapy system 10 to assist in managing, e.g., peripheral neuropathy or post-operative pain mitigation, ilioinguinal nerve therapy, intercostal nerve therapy, gastric drug induced stimulation for the treatment of gastric motility disorders and/or obesity, and muscle stimulation, or for mitigation of peripheral and localized pain (e.g., leg pain or back pain). As another example delivery site, catheter 18 may be positioned to deliver a therapeutic agent to a deep brain site or within the heart (e.g., intraventricular delivery of the agent) or blood vessels. Delivery of a therapeutic agent within the brain may help manage any number of disorders or diseases including, e.g., chronic pain, diabetes, depression or other mood disorders, dementia, obsessive-compulsive disorder, migraines, obesity, and movement disorders, such as Parkinson's disease, spasticity, and epilepsy. Catheter 18 may also be positioned to deliver insulin to a patient with diabetes.

As already mentioned, in some applications, therapy system 10 can be used to reduce pain experienced by patient 16. In such an application, IMD 12 can deliver one or more therapeutic agents to patient 16 according to one or more dosing programs different therapy parameters, such as specifying programmed doses, dose rates for the programmed doses, and therapy schedules that set forth specific times to deliver the programmed doses. The dosing programs may be a part of a program group for therapy, where the group includes a plurality of dosing programs and/or therapy schedules. In some examples, IMD 12 may be configured to deliver a therapeutic agent to patient 16 according to different therapy schedules on a selective basis. IMD 12 may include a memory to store one or more dosing programs and/or therapy schedules, instructions defining the extent to which patient 16 may adjust therapy parameters, switch between dosing programs, or undertake other therapy adjustments. Patient 16 or a clinician may select and/or generate additional dosing programs and/or therapy schedules for use by IMD 12 via external programmer 20 at any time during therapy or as designated by the clinician.

In some examples, multiple catheters 18 may be coupled to IMD 12 to target the same or different tissue or nerve sites within patient 16. Thus, although a single catheter 18 is shown in FIG. 1 for purposes of illustration, in other examples, system 10 may include multiple catheters or catheter 18 may define multiple lumens for delivering different therapeutic agents to patient 16 or for delivering a therapeutic agent to different tissue sites within patient 16. Accordingly, in some examples, IMD 12 may include a plurality of reservoirs for storing more than one type of therapeutic agent. In some examples, IMD 12 may include a single long tube that contains the therapeutic agent in place of a reservoir. However, for purposes of illustration, IMD 12 including a single reservoir is primarily discussed in this disclosure with reference to the example of FIG. 1.

Programmer 20 is an external computing device that is configured to communicate with IMD 12 by wireless telemetry. For example, programmer 20 may be a clinician programmer that the clinician uses to communicate with IMD 12 and program therapy delivered by the IMD. Alternatively, programmer 20 may be a patient programmer that allows patient 16 to view and modify therapy parameters associated with therapy programs. The clinician programmer may include additional or alternative programming features than the patient programmer. For example, more complex or sensitive tasks may only be allowed by the clinician programmer to prevent patient 16 from making undesired or unsafe changes to the operation of IMD 12.

Programmer 20 may be a hand-held computing device that includes a display viewable by the user and a user input mechanism that can be used to provide input to programmer 20. For example, programmer 20 may include a display screen (e.g., a liquid crystal display or a light emitting diode display) that presents information to the user. In addition, programmer 20 may include a keypad, buttons, a peripheral pointing device, touch screen, voice recognition, or another input mechanism that allows the user to navigate through the user interface of programmer 20 and provide input.

If programmer 20 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 20 may be a touch screen that allows the user to provide input directly to the user interface shown on the display. The user may use a stylus or their finger to provide input to the display.

In other examples, rather than being a handheld computing device or a dedicated computing device, programmer 20 may be a larger workstation or a separate application within another multi-function device. For example, the multi-function device may be a cellular phone, personal computer, laptop, workstation computer, or personal digital assistant that can be configured with an application to simulate programmer 20. Alternatively, a notebook computer, tablet computer, or other personal computer may enter an application to become programmer 20 with a wireless adapter connected to the personal computer for communicating with IMD 12.

When programmer 20 is configured for use by the clinician, programmer 20 may be used to transmit initial programming information to IMD 12. This initial information may include hardware information for system 10 such as the type of catheter 18, the position of catheter 18 within patient 16, the type and amount, e.g., by volume of therapeutic agent(s) delivered by IMD 12, a baseline orientation of at least a portion of IMD 12 relative to a reference point, therapy parameters of therapy programs stored within IMD 12 or within programmer 20, and any other information the clinician desires to program into IMD 12.

The clinician uses programmer 20 to program IMD 12 with one or more programs that define the therapy delivered by the IMD. During a programming session, the clinician may determine one or more dosing programs that may provide effective therapy to patient 16. Patient 16 may provide feedback to the clinician as to efficacy of a program being evaluated or desired modifications to the program. Once the clinician has identified one or more programs that may be beneficial to patient 16, the patient may continue the evaluation process and determine which dosing program and/or therapy schedule best alleviates the condition of the patient or otherwise provides efficacious therapy to the patient.

The dosing program and therapy schedule information may set forth therapy parameters, such as different predetermined dosages of the therapeutic agent (e.g., a dose amount), the rate of delivery of the therapeutic agent (e.g., rate of delivery of the fluid), the maximum acceptable dose, a time interval between successive supplemental boluses such as patient-initiated boluses (e.g., a lock-out interval), a maximum dose that may be delivered over a given time interval, and so forth. IMD 12 may include a feature that prevents dosing the therapeutic agent in a manner inconsistent with the dosing program. Programmer 20 may assist the clinician in the creation/identification of dosing programs by providing a methodical system of identifying potentially beneficial therapy parameters.

A dosage of a therapeutic fluid, such as a drug, may be expressed as an amount of drug, e.g., measured in milligrams or other volumetric units, provided to patient 16 over a time interval, e.g., per day or twenty-four hour period. In this sense, the dosage may also indicate a rate of delivery. This dosage amount may convey to the caregiver an indication of the probable efficacy of the drug and the possibility of side effects. In general, a sufficient amount of the drug should be administered in order to have a desired therapeutic effect, such as pain relief. However, the amount of the drug administered to the patient should be limited to a maximum amount, such as a maximum daily dose, in order to avoid potential side effects. Program information specified by a user via programmer 20 may be used to control the dosage amount, dosage rate, dosage time, maximum dose for a given time interval (e.g., daily), or other parameters associated with delivery of a drug or other fluid by IMD 12. Dosage may also prescribe particular concentrations of active ingredients in the therapeutic agent delivered by IMD 12 to patient 16.

In some cases, programmer 20 may also be configured for use by patient 16. When configured as the patient programmer, programmer 20 may have limited functionality in order to prevent patient 16 from altering critical functions or applications that may be detrimental to patient 16. In this manner, programmer 20 may only allow patient 16 to adjust certain therapy parameters or set an available range for a particular therapy parameter. In some cases, a patient programmer may permit the patient to control IMD 12 to deliver a supplemental, patient bolus, if permitted by the applicable therapy program administered by the IMD, e.g., if delivery of a patient bolus would not violate a lockout interval or maximum dosage limit. Programmer 20 may also provide an indication to patient 16 when therapy is being delivered, when IMD 12 needs to be refilled, or when the power source within programmer 20 or IMD 12 need to be replaced or recharged.

Whether programmer 20 is configured for clinician or patient use, programmer 20 may communicate to IMD 12 or any other computing device via wireless communication. Programmer 20, for example, may communicate via wireless communication with IMD 12 using radio frequency (RF) telemetry techniques. Programmer 20 may also communicate with another programmer or computing device via a wired or wireless connection using any of a variety of communication techniques including, e.g., RF communication according to the 802.11 or Bluetooth specification sets, infrared (IR) communication according to the IRDA specification set, or other standard or proprietary telemetry protocols. Programmer 20 may also communicate with another programming or computing device via exchange of removable media, such as magnetic or optical disks, or memory cards or sticks including, e.g., non-volatile memory. Further, programmer 20 may communicate with IMD 12 and another programmer via, e.g., a local area network (LAN), wide area network (WAN), public switched telephone network (PSTN), or cellular telephone network, or any other terrestrial or satellite network appropriate for use with programmer 20 and IMD 12.

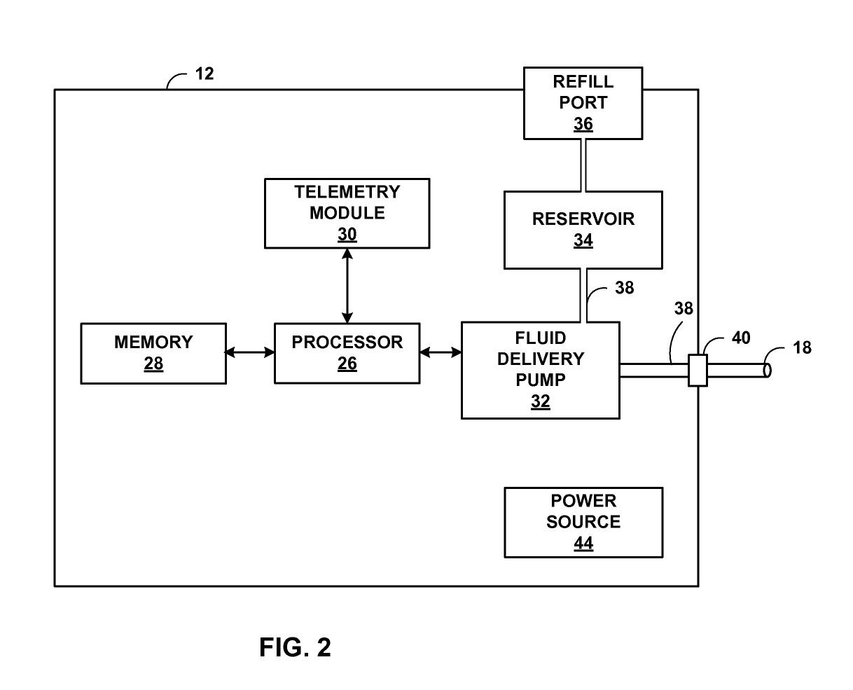

FIG. 2 is a functional block diagram illustrating components of an example of IMD 12, which includes processor 26, memory 28, telemetry module 30, fluid delivery pump 32, reservoir 34, refill port 36, internal tubing 38, catheter access port (CAP) 40, and power source 44. Processor 26 may be communicatively connected to memory 28, telemetry module 30, and control circuitry associated with fluid delivery pump 32. Fluid delivery pump 32 is connected to reservoir 34 and internal tubing 38. Reservoir 34 is connected to refill port 36. Catheter access port 40 is connected to internal tubing 38 and catheter 18. IMD 12 also includes power source 44, which is configured to deliver operating power to various components of the IMD.

During operation of IMD 12, processor 26 controls fluid delivery pump 32 with the aid of instructions associated with program information that is stored in memory 28 to deliver a therapeutic agent to patient 16 via catheter 18. In controlling fluid delivery pump 32, processor 26 may directly control circuitry associated with the fluid delivery pump to drive the pump. For example, the control circuitry may be responsive to instructions in the form of digital control values generated by processor 26 or analog control signals generated by a digital-to-analog converter (DAC) based on digital control values generated by processor 26. In either case, instructions executed by processor 26 may, for example, define dosing programs and/or therapy schedules that specify the amount of a therapeutic agent that is delivered to a target tissue site within patient 16 from reservoir 30 via catheter 18. The instructions may further specify the time at which the agent will be delivered and the time interval over which the agent will be delivered. The amount of the agent and the time over which the agent will be delivered are a function of, or alternatively determine, the dosage rate at which the fluid is delivered. The therapy programs may also include other therapy parameters, such as the frequency of bolus delivery, the type of therapeutic agent delivered if IMD 12 is configured to deliver more than one type of therapeutic agent, and so forth. Components described as processors within IMD 12, external programmer 20, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination.

Upon instruction from processor 26, fluid delivery pump 32 draws fluid from reservoir 34 and pumps the fluid through internal tubing 38 to catheter 18 through which the fluid is delivered to patient 16 to effect one or more of the treatments described above. Internal tubing 38 is a segment of tubing or a series of cavities within IMD 12 that run from reservoir 34, around or through fluid delivery pump 32 to CAP 40. In devices such as IMD 12, CAP 40 is often provided in addition to refill port 36 as a mechanism for percutaneous access to the device via, e.g., a hypodermic needle. Whereas the refill port 36 provides access to reservoir 34 to aspirate IMD 12 and refill the device with therapeutic fluid delivered to patient 16, CAP 40 includes a septum that provides direct access to catheter 18, thereby bypassing pump 32 and allowing a bolus of drug or fluid medication to be administered directly into the body of patient 16 at the site of the catheter via the CAP septum. CAP 40 may also be used as a diagnostic tool to troubleshoot the catheter or infusion problems. An example of an implantable drug pump having a CAP is shown in U.S. Pat. No. 6,293,922 by Haase, entitled “Apparatus And Method For Guiding And Limiting Access By Hypodermic Needles To Septum Of A Human Implantable Medical Treatment Device,” issued Sep. 25, 2001.

In examples described in this disclosure, IMD 12 includes at least one flexible member interposed between patient 16 and/or the control electronics of the device, e.g., processor 26, memory 28, telemetry module 30, power source 44, and the flow path of therapeutic fluid from pump 32. In one example, the flexible member includes the septum of CAP 40 (not shown in FIG. 2), which is configured as an accumulator to create compliance in the flow path of IMD 12 downstream from fluid delivery pump 32. By forming the septum of CAP 40 in a particular shape, as is described in greater detail below, the membrane can be configured to deform under pressure and then elastically recover, e.g., like a trampoline. In addition, the shape of the septum of CAP 40 may be designed with sufficient radial compression to seal after being punctured by a needle, which is generally a necessary attribute for allowing percutaneous access to catheter 18 via, e.g., a hypodermic needle.

Fluid delivery pump 32 can be any mechanism that delivers a therapeutic agent in some metered or other desired flow dosage to the therapy site within patient 16 from reservoir 30 via implanted catheter 18. In one example, fluid delivery pump 32 can be a squeeze pump that squeezes internal tubing 38 in a controlled manner, e.g., such as a peristaltic pump, to progressively move fluid from reservoir 34 to the distal end of catheter 18 and then into patient 16 according to parameters specified by a set of program information stored on memory 28 and executed by processor 26. Fluid delivery pump 32 can also be an axial pump, a centrifugal pump, a pusher plate, a piston-driven pump, or other means for moving fluid through internal tubing 38 and catheter 18. In one particular example, fluid delivery pump 32 can be an electromechanical pump that delivers fluid by the application of pressure generated by a piston that moves in the presence of a varying magnetic field and that is configured to draw fluid from reservoir 34 and pump the fluid through internal tubing 38 and catheter 18 to patient 16.

Periodically, fluid may need to be supplied percutaneously to reservoir 34 because all of a therapeutic agent has been or will be delivered to patient 16, or because a clinician wishes to replace an existing agent with a different agent or similar agent with different concentrations of therapeutic ingredients. In a similar fashion as the septum of CAP 40, refill port 26 can therefore comprise a self-sealing membrane to prevent loss of therapeutic agent delivered to reservoir 30 via refill port 26. For example, after a percutaneous delivery system including a hypodermic needle penetrates the membrane of refill port 26, the membrane may seal shut when the needle is removed from refill port 26.

Although controlling delivery of a therapeutic fluid to patient 16, e.g., according to a dosing program and/or therapy schedule, has been described as executed by IMD 12, and in particular, processor 26, in other examples, one or more of these functions may be carried out by other devices including, e.g., external programmer 20. For example, one or more aspects of device control of IMD 12 may be executed by a processor of and stored on a memory of programmer 20 and/or a processor and memory of another electronic device communicatively connected to IMD 12.

Memory 28 of IMD 12 may store program information including instructions for execution by processor 26 or a processor of another device, such as, but not limited to, therapy programs, historical therapy programs, therapy schedules defining timing for delivery of fluid from reservoir 34 to catheter 18, and any other information regarding therapy of patient 16. A program may indicate the dosage of therapeutic fluid, and processor 26 may accordingly deliver therapy. Memory 28 may include separate memories for storing instructions, patient information, therapy parameters (e.g., grouped into sets referred to as dosing programs and/or therapy schedules), therapy adjustment information, program histories, and other categories of information such as any other data that may benefit from separate physical memory modules. Therapy adjustment information may include information relating to timing, frequency, rates and amounts of patient boluses or other permitted patient modifications to therapy. In some examples, memory 28 stores program instructions that, when executed by processor 26, cause IMD 12 and processor 26 to perform the functions attributed to them in this disclosure.

At various times during the operation of IMD 12 to treat patient 16, communication to and from IMD 12 may be necessary to, e.g., change therapy programs, adjust parameters within one or more programs, configure or adjust a particular bolus, or to otherwise download information to or from IMD 12. Processor 26 therefore controls telemetry module 30 to wirelessly communicate between IMD 12 and other devices including, e.g. programmer 20. Telemetry module 30 in IMD 12, as well as telemetry modules in other devices described in this disclosure, such as programmer 20, can be configured to use RF communication techniques to wirelessly send and receive information to and from other devices respectively. In addition, telemetry module 30 may communicate with programmer 20 via proximal inductive interaction between IMD 12 and the external programmer. Telemetry module 30 may send information to external programmer 20 on a continuous basis, at periodic intervals, or upon request from the programmer.

Power source 44 delivers operating power to various components of IMD 12. Power source 44 may include a small rechargeable or non-rechargeable battery and a power generation circuit to produce the operating power. In the case of a rechargeable battery, recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, rechargeable or non-rechargeable batteries may be used for a limited period of time. As another alternative, an external inductive power supply could transcutaneously power IMD 12 as needed or desired.

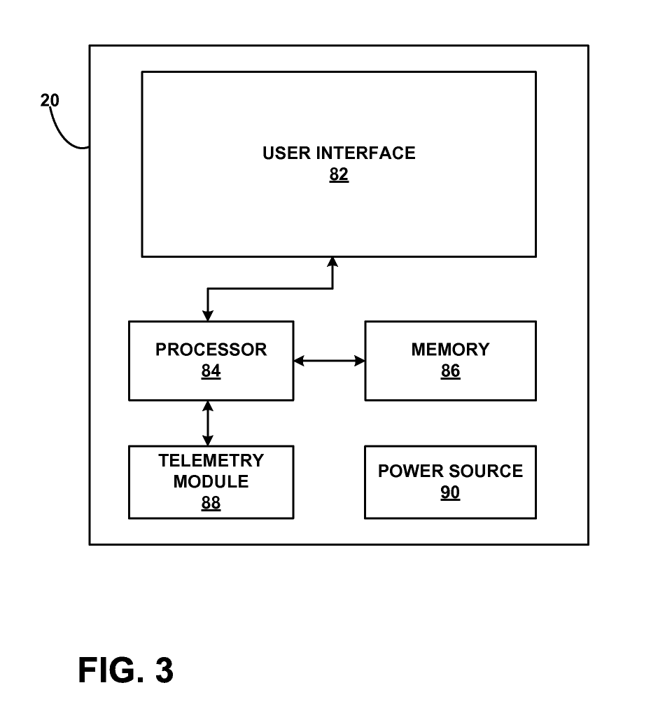

FIG. 3 is a functional block diagram illustrating various components of external programmer 20 for IMD 12. As shown in FIG. 3, external programmer 20 includes user interface 82, processor 84, memory 86, telemetry module 88, and power source 90. A clinician or patient 16 interacts with user interface 82 in order to manually change the parameters of a dosing program, change dosing programs within a group of programs, view therapy information, view historical therapy regimens, establish new therapy regimens, or otherwise communicate with IMD 12 or view or edit programming information.

User interface 82 may include a screen and one or more input buttons, as discussed in greater detail below, that allow external programmer 20 to receive input from a user. Alternatively, user interface 82 may additionally or only utilize a touch screen display, as in the example of clinician programmer 60. The screen may be a liquid crystal display (LCD), dot matrix display, organic light-emitting diode (OLED) display, touch screen, or any other device capable of delivering and/or accepting information. For visible indications of therapy program parameters or operational status, a display screen may suffice. For audible and/or tactile indications of program parameters or operational status, programmer 20 may further include one or more audio speakers, voice synthesizer chips, piezoelectric buzzers, or the like.

Input buttons for user interface 82 may include a touch pad, increase and decrease buttons, emergency shut off button, and other buttons needed to control the therapy, as described above with regard to programmer 20. Processor 84 controls user interface 82, retrieves data from memory 86 and stores data within memory 86. Processor 84 also controls the transmission of data through telemetry module 88 to IMD 12. The transmitted data may include therapy program information specifying various drug delivery program parameters. Memory 86 may include operational instructions for processor 84 and data related to therapy for patient 16.

User interface 82 may be configured to present therapy program information to the user. User interface 82 enables a user to program IMD 12 in accordance with one or more dosing programs, therapy schedules, or the like. For example, a user such as a clinician, physician or other caregiver may input patient information, drug information, therapy schedules, priming information, bridging information, drug/IMD implant location information, or other information to programmer 20 via user interface 82. In addition, user interface 82 may display therapy program information as graphical bar graphs or charts, numerical spread sheets, or in any other manner in which information may be displayed. Further, user interface 82 may present nominal or suggested therapy parameters that the user may accept via user interface 82.

Telemetry module 88 allows the transfer of data to and from IMD 12. Telemetry module 88 may communicate automatically with IMD 12 at a scheduled time or when the telemetry module detects the proximity of IMD 12. Alternatively, telemetry module 88 may communicate with IMD 12 when signaled by a user through user interface 82. To support RF communication, telemetry module 88 may include appropriate electronic components, such as amplifiers, filters, mixers, encoders, decoders, and the like.

Power source 90 may be a rechargeable battery, such as a lithium ion or nickel metal hydride battery. Other rechargeable or conventional batteries may also be used. In some cases, external programmer 20 may be used when coupled to an alternating current (AC) outlet, i.e., AC line power, either directly or via an AC/DC adapter.

In some examples, external programmer 20 may be configured to recharge IMD 12 in addition to programming the device. Alternatively, a recharging device may be capable of communication with IMD 12. Then, the recharging device may be able to transfer programming information, data, or any other information described herein to IMD 12. In this manner, the recharging device may be able to act as an intermediary communication device between external programmer 20 and IMD 12.

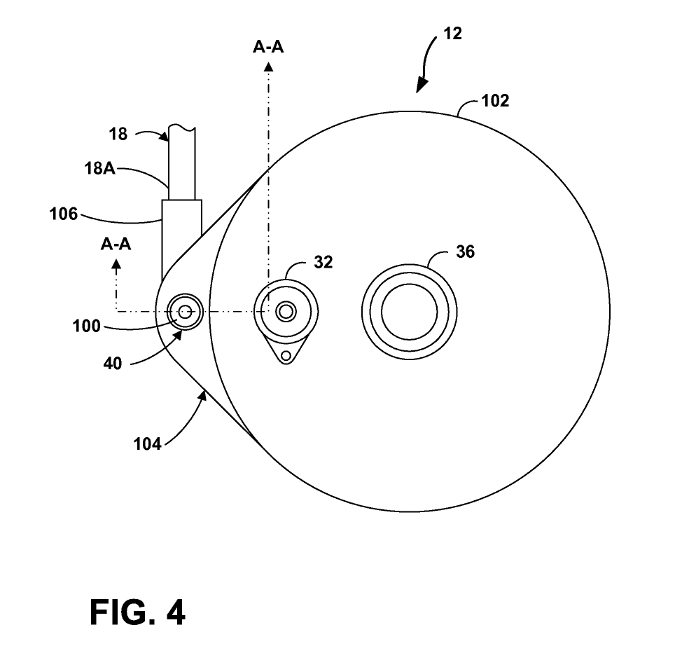

FIG. 4 is a plan view of an example configuration of IMD 12 including catheter 18, fluid delivery pump 32, refill port 36, CAP 40, and CAP septum 100. IMD 12 includes housing 102 and header 104, which includes CAP 40 and CAP septum 100. In another example, however, CAP 40 and septum 100 may be arranged in another location in housing 102 of IMD 12. As described above, fluid delivery pump 32 is connected to CAP 40 by internal tubing 38 (not shown in FIG. 4). Fluid delivery pump 32 is also connected to catheter extension 106 via tubing 38 in header 104. Catheter extension 106 is connected to proximal end 18A of catheter 18, from which the catheter extends from IMD 12 to the target delivery site within patient 16. As fluid delivery pump 32 delivers pulses of therapeutic fluid through catheter extension 106 and catheter 18 to patient 16, CAP septum 100 is configured to deform to absorb at least a portion of the initial pressure spike induced by the fluid pulse. CAP septum 100 may deform such that a portion of the CAP septum moves away from a normal position and/or shape in response to delivery of a fluid pulse, and then elastically recovers to the normal position and or shape as the fluid pulse subsides. The compliance created in CAP septum 100 reduces the energy required for the pump to deliver fluid to patient 16, which, in turn, increases the longevity of IMD 12.

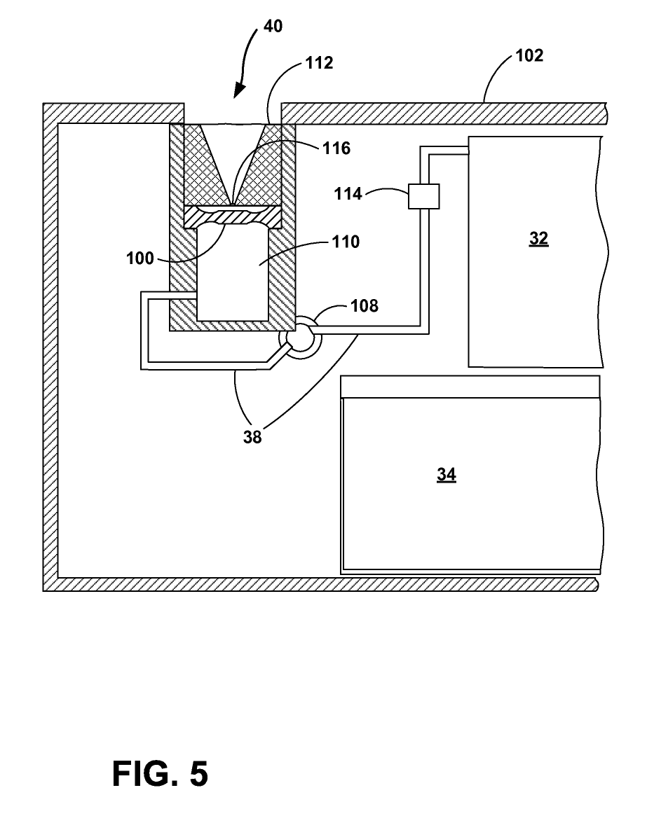

FIG. 5 is a section view of CAP 40 cut along section line A-A of FIG. 4. CAP 40 includes septum 100, discharge outlet 108, well 110, and needle guide 112. CAP 40 provides a sealed structure through which fluid may be directly passed to catheter 18 (FIGS. 1-2), the distal end of which 18A is connected to discharge outlet 108. In particular, well 110 is in fluid communication with discharge outlet 108 via, e.g., internal tubing 38. Well 110 is accessible from an exterior side of housing 102 of IMD 12 through needle guide 112 and septum 100. Therapeutic fluid may be delivered, e.g., via a hypodermic needle through septum 100 to well 110, from which the fluid travels through tubing 38 and discharge outlet 108 to catheter 18. In some examples, septum 100 may also act to seal well 110 from contaminants.

As illustrated in the example of FIG. 5, discharge outlet 108 of CAP 40 may also define an opening that fluidically couples catheter 18 to fluid within reservoir 34 via internal tubing 38. IMD 12 may also include metering mechanism 114 to control flow of therapeutic fluid from pump 32 to discharge outlet 108. Metering mechanism 114 may, in some examples, include a valve, e.g. a check valve that permits flow of the therapeutic fluid from pump 32 through tubing 38 to discharge outlet 108, but prevents back flow from the valve to the pump.

Needle guide 112 is generally formed as a conical depression for guiding a hypodermic needle into CAP septum 100. Needle guide 112 includes an aperture 116 at the center for limiting the maximum needle diameter that can be used to access CAP 40. The size of aperture 116 may act to prevent access by needles having a diameter greater than the diameter of the hole. The conical depression of needle guide 112 may have a smooth surface and an angle such that needles that are inserted into the conical depression misaligned with aperture 116 will be guided to the opening without damaging the tip of the hypodermic needle. It is noted that, in order to more clearly illustrate CAP septum 100 in FIG. 4, needle guide 112 is not shown in that view.

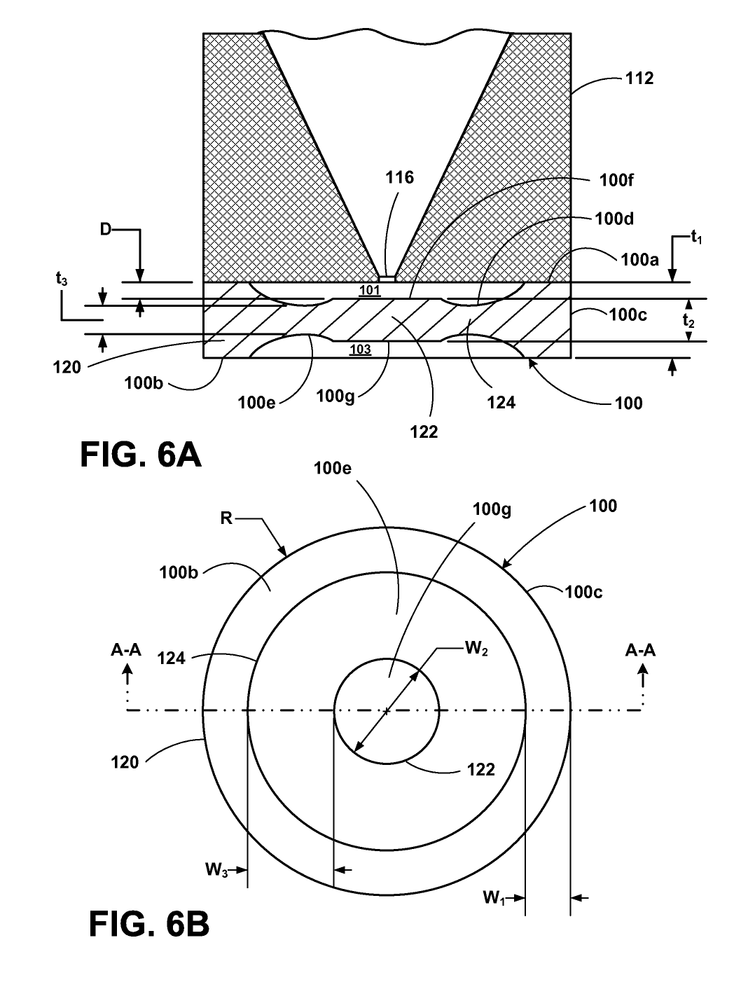

FIGS. 6A and 6B are detail views of CAP 40. FIG. 6A is a section view of CAP 40 including septum 100 and needle guide 112 cut along section line A-A of FIGS. 4 and 6B. FIG. 6B is a plan view of CAP 40 including septum 100 from below the septum. Needle guide 112 abuts one side of septum 100. In the orientation illustrated in FIGS. 5 and 6, for example, needle guide 112 abuts the top surface of septum 100.

CAP septum 100 is generally formed as a disc including two major circular surfaces connected by an annular minor surface. Septum 100 also includes material voids 101 and 103 formed in each of the two major surfaces of the disc-shaped septum to form rim 120, which defines a perimeter portion of septum 100, hub 122, which defines a central portion of the septum, and web 124, which defines an intermediary portion of the septum. Rim 120 is an outermost, raised ring portion configured to be secured to a structure, e.g., needle guide 112 and includes a first thickness, t₁. Rim 120 is generally bounded on the top and bottom of septum 100 by surfaces 100a and 100b, respectively, and circumferentially by annular surface 100c. Hub 122 is a central disc-like portion that is surrounded by rim 120 and includes a second thickness, t₂, which is less than the first thickness, t₁, of rim 120. Web 124 is an intermediate, recessed ring portion that connects rim 120 to hub 122 and includes a third thickness, t₃, which is less than the the second thickness, t₂, of hub 122. Web 124 is formed by channels 100d, 100e, while hub 122 is formed by surfaces 100f and 100g. Channels 100d, 100e of web 124 are formed as rings that, in the example of FIG. 6, are substantially concentric with surfaces 100a, 100b of rim 120. Surfaces 100f and 100g of hub 122 are offset from surfaces 100a, 100b, respectively, of rim 120 by a distance D. In some examples, the distance between surface 100f of hub 122 and surface 100a of rim 120 may be different than the distance between surface 100g of hub 122 and surface 100b of rim 120. Hub 122 including surfaces 100f and 100g and web 124 including channels 100d, 100e generally form material voids 101, 103, respectively in septum 100 to facilitate deformation of the septum under pressure from the therapeutic fluid flowing through IMD 12.

Rim 120, hub 122, and web 124 each have lateral widths W₁, W₂, and W₃ respectively. In the example of FIGS. 6A and 6B, the lateral width, W2, of hub 122 corresponds to the diameter of the disc-like portion of septum 100. Additionally, septum 100 has an overall radius, R. In some examples, the thickness, t₁, of rim 120 is in a range from approximately 2 millimeters (0.08 inches) to approximately 4 millimeters (0.16 inches) and the lateral width, W₁, of the rim is in a range from approximately 1 millimeter (0.04 inches) to approximately 2.6 millimeters (0.1 inches). Additionally, in some examples, the thickness, t₂, of hub 122 is in a range from approximately 1 millimeter (0.04 inches) to approximately 3 millimeters (0.12 inches) and the lateral width, W₂, of the hub is in a range from approximately 2 millimeters (0.08 inches) to approximately 3 millimeters (0.12 inches). In some examples, the thickness, t₃, of web 124 is in a range from approximately 1 millimeter (0.04 inches) to approximately 2 millimeters (0.08 inches) and the lateral width, W₃, of the web is in a range from approximately 2 millimeters (0.08 inches) to approximately 4.6 millimeters (0.18 inches). Finally, in some examples, the radius, R, of septum 100 is in a range from approximately 5 millimeters (0.2 inches) to approximately 9.7 millimeters (0.38 inches).

The material voids 101, 103 formed by hub 122 and web 124 in septum 100 permits the septum to deform under pressure from the therapeutic fluid flowing through IMD 12. In particular, in the example of FIGS. 4-6B, pump 32 of IMD 12 may deliver a dose of fluid to patient 16 via internal tubing 38 and catheter 18 in the form of a fluid pulse. The fluid pulse delivered by pump 32 acts to induce an initial pressure spike in the flow path of IMD 12. Mechanically overcoming the pressure spike increases the power consumption of pump 32, which, in turn, may reduce the longevity of IMD 12 due to depletion of stored power in a battery. CAP septum 100, therefore, is configured to create compliance in the flow path of IMD 12 to at least partially absorb the initial pressure spike induced by the fluid pulse delivered by pump 32 by deflecting and bulging toward needle guide 112 into the material void 101 formed by hub 122 and web 124. The compliance created in CAP septum 100 may reduce the energy required for pump 32 to deliver fluid to patient 16, which, in turn, may act to increase the longevity of IMD 12. Reducing energy, in turn, may act to extend the longevity of IMD 12 or maintain longevity while reducing the size of pump 32.

Material void 101 formed by hub 122 and web 124 in septum 100 may become partially or completely filled with fluids from the body of patient 16. However, provided aperture 116 in needle guide 112 remains substantially unimpeded, any body fluids in void 101 should flow freely out of the aperture and therefore should not substantially affect the ability of CAP septum 100 to deform under pressure from the therapeutic fluid flowing through IMD 12. However, in some examples, needle guide 112 or another component of IMD 12 may include additional apertures in fluid communication with material void 101 in order guard against the possibility of aperture 116 becoming partially or completely blocked. Additionally, the size of aperture 116 may be varied to reduce the likelihood of blockages forming.

In other examples of IMD 12, a CAP septum may be formed in shapes other than the disc shape of septum 100 of FIGS. 6A and 6B and may include channels, concavities, or other material voids having various configurations other than the material void formed by hub 122 and web 124 in septum 100. In such examples, the CAP septum may generally include a material void that creates an offset between the septum and adjacent structure of IMD 12 such that the septum may deform under pressure in order to create compliance in the flow path of IMD 12 to completely, or at least partially, absorb the initial pressure spike induced by a fluid pulse delivered by pump 32.

In some examples, the CAP septum may include a material void in only one of two major surfaces of the septum. In particular, the septum may include a material void in a surface adjacent to structure of the IMD such that the void in the septum creates an offset between the septum and the structure of the IMD such that the septum may deform under pressure. In the example of FIGS. 6A and 6B, septum 100 may therefore include channel 100d and surface 100f offset from surface 100a, while not including channel 100e and surface 100g such that a material void is formed in only the side of the septum adjacent needle guide 112. Additionally, although rim 120, hub 122, and web 124 that form septum 100 have three variations in thickness, t₁, t₂, and t₃, respectively, in other examples, the CAP septum may have a material void that generally forms only two variations in thickness between the constrained perimeter and the central portion of the septum that is configured to deform under pressure. For example, instead of including web 124 and hub 122 at two different thicknesses, septum 100 may, in some examples, include a single concavity that generally forms a uniform thickness across the central portion of the septum that is less than rim 120, i.e. the constrained perimeter portion.

As described above, example septa of the CAP of a fluid delivery device may be constructed from a variety of biocompatible elastomeric materials including, e.g., silicone, fluoroelastomers, perfluoroelastomers and the like. The material from which the septum is formed may include specific mechanical properties including, e.g., specific values for durometer. In some examples, the septum may be formed of a material having a durometer in a range from approximately 50 to approximately 80. In one example, the septum may be formed of a material having a durometer in a range from approximately 50 to approximately 65. In some examples, the silicone or other material from which the CAP septum is fabricated may have tear resistant properties, e.g. to enhance the reseal properties of the septum by inhibiting needles from permanently gouging the CAP septum.

A reduction in the energy to pump a therapeutic fluid from a fluid delivery device to a patient can be realized if the flow path downstream of the pumping mechanism has some compliance. In this context, compliance means that the flow path is flexible or non-rigid. In hydraulic systems, a component that creates compliance may be referred to as an accumulator. By shaping a CAP septum of a fluid delivery device as described in the foregoing examples, the septum may be made to flex under pressure like a trampoline. In addition, the shape and material composition of the septum provide sufficient radial compression to seal after a needle stick, which is an important design attribute for CAP septa. Configuring and employing a CAP septum in accordance with the foregoing examples may act to provide several advantages including reducing the cost of the fluid delivery device in which the septum is included, reducing the occurrence of flow path accumulator fatigue based failures, reducing the number of parts in and size of the device, and increasing device longevity by reducing the power required to drive the device pump.

Various examples have been described. These and other examples are within the scope of the following claims.

1. A therapeutic fluid delivery system comprising:
a fluid delivery device configured to deliver a therapeutic fluid to a patient; and
a catheter access port (CAP) septum connected to the fluid delivery device;
wherein the CAP septum is configured as an accumulator for flow of the therapeutic fluid through the fluid delivery device.

2. The system of claim 1, wherein the CAP septum comprises an elastomeric material.

3. The system of claim 1, wherein the CAP septum comprises at least one material void in at least one major surface of the CAP septum, the material void configured to facilitate deformation of the CAP septum under pressure from the therapeutic fluid.

4. The system of claim 1, wherein the CAP septum comprises a disc comprising two circular major surfaces connected by a minor annular surface.

5. The system of claim 4, wherein the CAP septum comprises at least one channel formed in one of the two circular major surfaces as a ring substantially concentric with the one of the two circular major surfaces.

6. The system of claim 4, wherein the CAP septum comprises at least one channel formed in each of the two circular major surfaces, wherein each channel is formed as a ring substantially concentric with the one of the two circular major surfaces in which it is formed.

7. The system of claim 1, wherein the CAP septum comprises a disc comprising a rim comprising a first thickness, a hub comprising a second thickness less than the first thickness, and web connecting the hub to the rim and comprising a third thickness less than the second thickness.

8. A catheter access port (CAP) septum for a therapeutic fluid delivery device comprising:
a rim configured to be secured to a structure and comprising a first thickness;
a hub surrounded by the rim and comprising a second thickness that is less than the first thickness of the rim; and
a web connecting the rim to the hub and comprising a third thickness that is less than the second thickness of the hub.

9. The system of claim 8, wherein the CAP septum comprises a disc comprising two circular major surfaces connected by a minor annular surface.

10. The system of claim 9, wherein the web comprises at least one channel formed in one of the two circular major surfaces as a ring substantially concentric with the one of the two circular major surfaces.

11. The system of claim 9, wherein the web comprises at least one channel formed in each of the two circular major surfaces, wherein each channel is formed as a ring substantially concentric with the one of the two circular major surfaces in which it is formed.

12. A catheter access port (CAP) septum for a therapeutic fluid delivery device comprising:
a disc comprising two circular major surfaces connected by a minor annular surface; and
at least one material void formed in at least one of the two circular major surfaces and configured to facilitate deformation of the CAP septum under pressure from a therapeutic fluid in a flow path of the fluid delivery device.

13. The CAP septum of claim 12, wherein the at least one material void comprises at least one channel formed in one of the two circular major surfaces as a ring substantially concentric with the one of the two circular major surfaces.

14. The CAP septum of claim 12, wherein the at least one material void comprises at least one channel formed in each of the two circular major surfaces, wherein each channel is formed as a ring substantially concentric with the one of the two circular major surfaces in which it is formed.

15. The CAP septum of claim 14, wherein the disc and the at least one channel formed in each of the two circular major surfaces forms:
a rim configured to be secured to a structure and comprising a first thickness;
a hub surrounded by the rim and comprising a second thickness that is less than the first thickness of the rim; and
a web connecting the rim to the hub and comprising a third thickness that is less than the second thickness of the hub.

16. A therapeutic fluid delivery device comprising:
a pump configured to deliver a therapeutic fluid to a patient via a flow path;
a processor programmable to cause the pump to deliver the therapeutic fluid to the patient; and
a catheter access port (CAP) septum arranged along the flow path and comprising at least one material void configured to facilitate deformation of the CAP septum under pressure from the therapeutic fluid in the flow path.

17. The device of claim 16, wherein the CAP septum comprises an elastomeric material.

18. The device of claim 16, wherein the CAP septum comprises a disc comprising two circular major surfaces connected by a minor annular surface.

19. The device of claim 18, wherein the at least one material void comprises at least one channel formed in one of the two circular major surfaces as a ring substantially concentric with the one of the two circular major surfaces.

20. The device of claim 18, wherein the at least one material void comprises at least one channel formed in each of the two circular major surfaces, wherein each channel is formed as a ring substantially concentric with the one of the two circular major surfaces in which it is formed.

21. The device of claim 16, wherein the CAP septum comprising the disc comprising two circular major surfaces connected by a minor annular surface and the at least one channel formed in each of the two circular major surfaces forms:
a rim configured to be secured to a structure and comprising a first thickness;
a hub surrounded by the rim and comprising a second thickness that is less than the first thickness of the rim; and
a web connecting the rim to the hub and comprising a third thickness that is less than the second thickness of the hub.
